(19) United States
(12) Patent Application Publication
(10) Pub. No.: US 2018/0161023 A1
(43) Pub. Date: Jun. 14, 2018

(54) DUAL-PURPOSE BIOPSY PUNCH

(71) Applicant: Mohammed Saud ALSAIDAN, Riyadh (SA)

(72) Inventor: Mohammed Saud ALSAIDAN, Riyadh (SA)

(21) Appl. No.: 15/378,758

(22) Filed: Dec. 14, 2016

(51) Int. Cl. A61B 10/02; A61B 17/3205; A61M 1/00

(52) U.S. Cl. CPC A61B 10/0283; A61B 17/32053; A61B 2017/320064; A61B 2010/0208; A61M 1/007

(57) ABSTRACT

A biopsy punch includes a hollow longitudinal shaft with an inner perimeter proximal restriction point and an inner perimeter distal restriction point; a tubular blade affixed to a distal end of the hollow longitudinal shaft, wherein the tubular blade forms a first chamber; a plunger configured to slide within the hollow longitudinal shaft; a withdrawer/ejector housed within the plunger; a withdrawer/ejector piston affixed to a distal end of the withdrawer/ejector; a restriction piston affixed around a perimeter of the plunger; a second chamber formed within the plunger between a distal end of the plunger and the withdrawer/ejector piston; and a hollow needle configured to move into and out of the first chamber via the plunger, wherein the hollow needle is affixed at a proximal end to the distal end of the plunger, and wherein the proximal end of the needle extends into the second chamber.

### BACKGROUND

### Grant of Non-Exclusive Right

This application was prepared with financial support from the Saudi Arabian Cultural Mission, and in consideration therefore the present inventor(s) has granted The Kingdom of Saudi Arabia a non-exclusive right to practice the present invention in the United States.

### Background

A biopsy punch is an instrument used for obtaining a tissue specimen for histopathological examination. It is also used for excising tissue and for hair transplantation. Most organs can be biopsied. However, punches are commonly used to take biopsies of skin, nails, and hair.

A basic biopsy punch includes a handle and a cutting member, which can be made of steel. The cutting member is generally a hollow cylinder that is sharp at a lower edge. The diameter of the lower edge can vary depending upon the type of biopsy to be obtained. In an embodiment, the diameter of the cutting member is in a range of 1-8 mm and a height of the cutting member is in a range of 2-20 mm.

The “background” description provided herein is for the purpose of generally presenting the context of the disclosure. Work of the presently named inventors, to the extent it is described in this background section, as well as aspects of the description which may not otherwise qualify as conventional at the time of filing, are neither expressly nor impliedly admitted as conventional against the present disclosure.

### SUMMARY

In one embodiment, a biopsy punch includes a hollow longitudinal shaft including an inner perimeter proximal restriction point and an inner perimeter distal restriction point; a tubular blade affixed to a distal end of the hollow longitudinal shaft, wherein the tubular blade forms a first chamber; a plunger configured to slide within the hollow longitudinal shaft; a withdrawer/ejector housed within the plunger; a withdrawer/ejector piston affixed to a distal end of the withdrawer/ejector; a restriction piston affixed around a perimeter of the plunger, wherein the restriction piston is configured to limit movement of the plunger at the proximal restriction point and the distal restriction point; a second chamber formed within the plunger between a distal end of the plunger and the withdrawer/ejector piston, wherein contents of the second chamber are drawn and aspirated via the withdrawer/ejector; and a hollow needle configured to move into and out of the first chamber via the plunger, wherein the hollow needle is affixed at a proximal end to the distal end of the plunger, and wherein the proximal end of the needle extends into the second chamber.

The foregoing paragraphs have been provided by way of general introduction, and are not intended to limit the scope of the following claims. The described embodiments will be best understood by reference to the following detailed description taken in conjunction with the accompanying drawings.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings, wherein:

FIG. 1 is a cross-sectional view of a biopsy punch in a biopsy retrieval position according to one embodiment;

FIG. 2 is a cross-sectional view of a biopsy punch in a biopsy release position according to one embodiment;

FIG. 3 is a cross-sectional view of a biopsy punch in a position in which a cytopathology specimen is ejected according to one embodiment;

FIG. 4 is a cross-sectional detailed view of a biopsy needle according to one embodiment;

FIG. 5A is a cross-sectional view of a normal skin tissue according to one embodiment;

FIG. 5B is a cross-sectional view of a vesicle formed between an epidermis external layer and a dermis internal layer according to one embodiment;

FIG. 5C is a cross-sectional view of a tubular blade of a biopsy punch penetrating through a vesicle according to one embodiment;

FIG. 6 is a flowchart illustrating a method of using a biopsy punch according to one embodiment; and

FIG. 7 is a flowchart illustrating a method of obtaining a biopsy specimen according to one embodiment.

### DETAILED DESCRIPTION

The following descriptions are meant to further clarify the present disclosure by giving specific examples and embodiments of the disclosure. These embodiments are meant to be illustrative rather than exhaustive. The full scope of the disclosure is not limited to any particular embodiment disclosed in this specification, but rather is defined by the claims.

In the interest of clarity, not all of the features of the implementations described herein are shown and described in detail. It will be appreciated that in the development of any such actual implementation, numerous implementation-specific decisions need to be made in order to achieve the developer's specific goals, such as compliance with application- and business-related constraints, and that these specific goals will vary from one implementation to another and from one developer to another.

In the following descriptions, and for the sake of clarification, proximal end means the end of a biopsy punch closer to the hand of a person holding the biopsy punch and performing an associated procedure. Distal end means the end of the biopsy punch farther away from the person's hand using the biopsy punch and closer to the biopsy point on the patient or object undergoing the biopsy procedure.

A biopsy punch can be used to obtain a histopathology specimen. Histopathology refers to examination of a biopsy after the specimen has been processed. The tissue is removed from a body or plant and placed into a fixative that stabilizes the tissue to prevent decay. A commonly used fixative is formaldehyde in water. The tissue is then prepared for viewing under a microscope.

Embodiments described herein include a biopsy punch that can also be used to obtain a cytopathology specimen. Cytopathology is a branch of pathology that studies and diagnoses diseases on a cellular level. It is generally used on samples of free cells or tissue fragments. A cytopathology specimen can be smeared across a glass microscope slide in a smear test for subsequent staining and microscopic examination. Cytology samples can also include other forms of preparation, such as cyto-centrifugation.

Embodiments described herein provide a biopsy punch configured to simultaneously collect a histopathology specimen and a cytopathology specimen in a single biopsy procedure. Separate chambers are provided in the biopsy punch for collection and dissemination of each type of specimen.

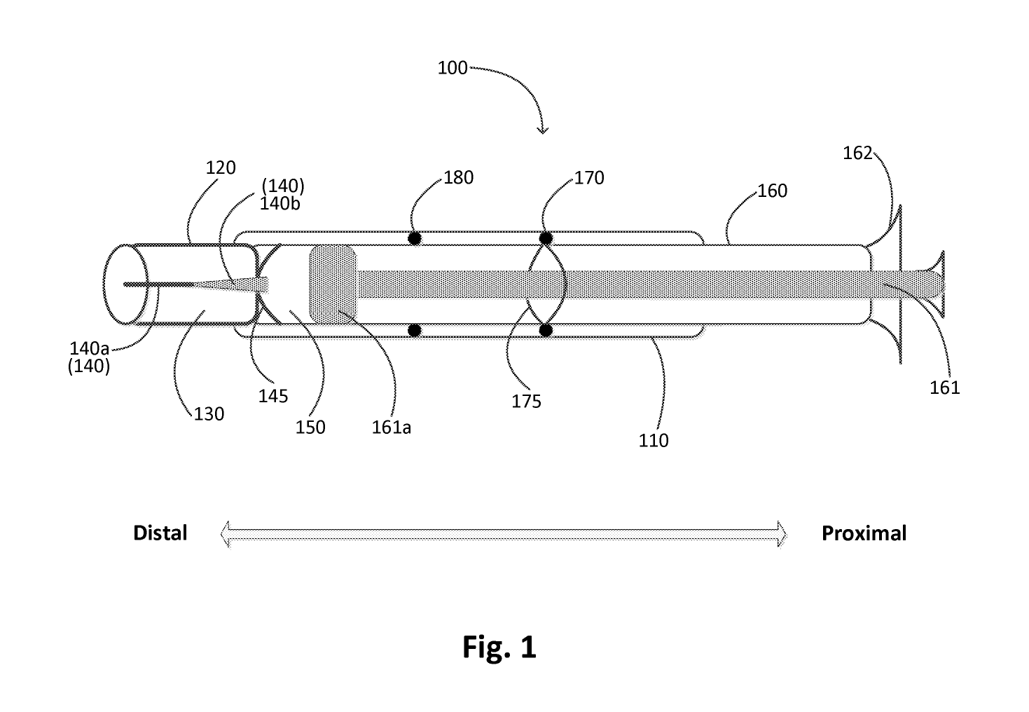

FIG. 1 is a cross-sectional view of a biopsy punch 100 in accordance with embodiments described herein. The biopsy punch 100 includes a punch body 110, which serves as a handle during the biopsy procedure and is configured to house other constituent components. A tubular blade 120 is attached to a distal end of the longitudinal axis of the punch body 110. The tubular blade 120 is generally cylindrical in shape to match the configuration of the punch body 110. However, other geometries for the tubular blade 120 and the punch body 110 are contemplated by embodiments described herein. The distal end of the tubular blade 120 has a sharp edge and is configured to cut through body or plant tissue for the purpose of obtaining a biopsy sample.

A histopathology specimen chamber 130 resides within the inner walls of the tubular blade 120. When the tubular blade 120 cuts through the body or plant tissue, the cut tissue is forced into the histopathology specimen chamber 130.

A plunger body 160 fits within the punch body 110 and is configured to slide within the punch body 110. A punch flanged end 162 provides a mechanism of sliding the plunger body 160 within the punch body 110. A cytopathology specimen chamber 150 is positioned at a plunger distal end 145 within the plunger body 160. A needle 140 is attached at a proximal end to the plunger distal end 145. The plunger body 160, the cytopathology specimen chamber 150, the plunger distal end 145, and the needle 140 slide together within the punch body 110 of the biopsy punch 100.

FIG. 1 illustrates a smooth needle portion 140a on a distal portion of the needle 140, and a non-smooth roughened needle portion 140b on a proximal portion of the needle 140. The smooth needle portion 140a provides a sharp piercing end that will easily penetrate through a tissue sample during a biopsy retrieval procedure.

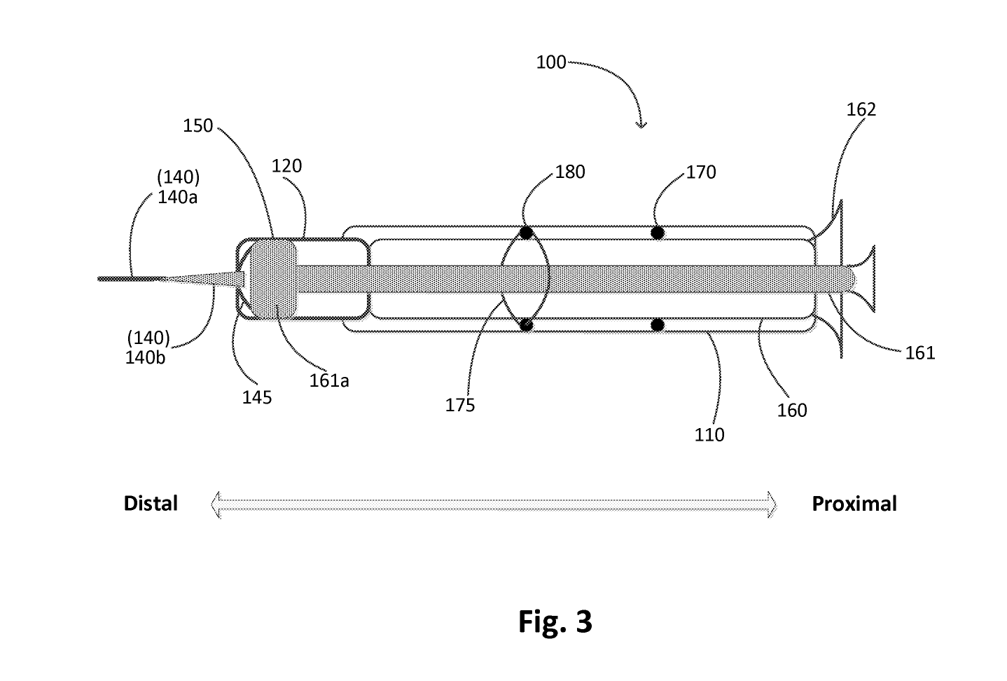

The needle 140 is centrally positioned within the histopathology specimen chamber 130 when the biopsy punch 100 is positioned in a biopsy retrieval position, as illustrated in FIG. 1. The needle 140 is configured to obtain a cytopathology specimen when biopsy tissue is obtained within the histopathology specimen chamber 130. As a result, a histopathology specimen is obtained within the histopathology specimen chamber 130 and a cytopathology specimen is obtained within the needle 140 during the same biopsy retrieval procedure. The needle 140 and the cytopathology specimen chamber 150 are described in more detail herein with reference to FIG. 3.

A restriction piston 175 is positioned around an outer perimeter of the plunger body 160. A proximal restriction point 170 is positioned within the inner perimeter of the punch body 110, such that the restriction piston 175 limits the plunger body 160 from moving past the proximal restriction point 170 and exposing the needle 140 beyond the tubular blade 120 in a biopsy retrieval position. The distal end of the needle 140 is in a near vicinity to the distal end of the tubular blade 120 in order to simultaneously obtain a histopathology specimen and a cytopathology specimen.

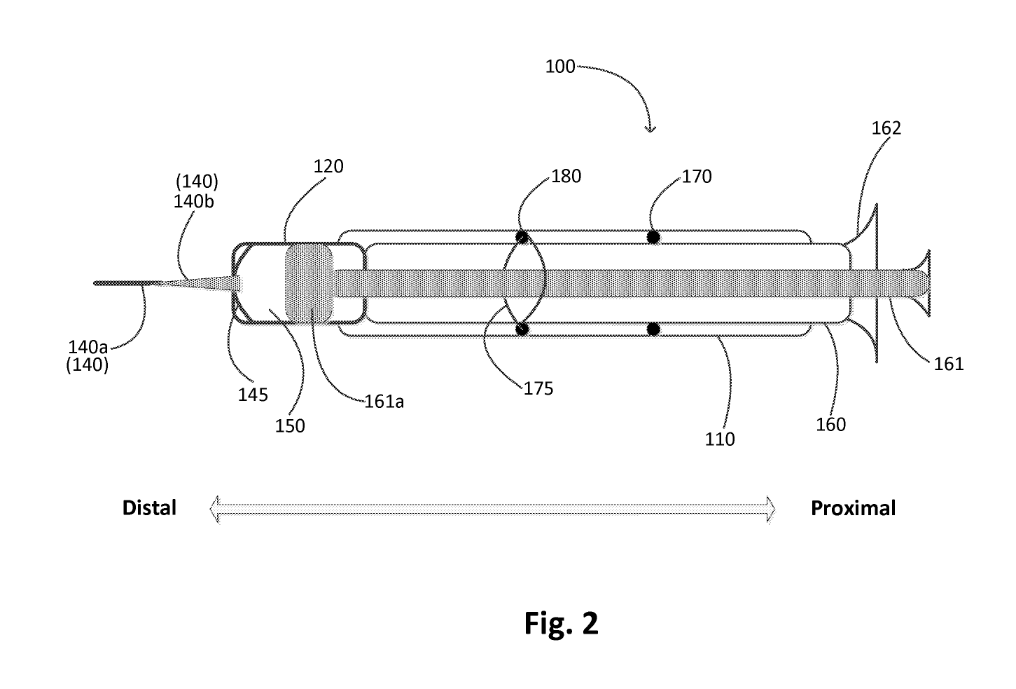

The restriction piston 175 is positioned at the proximal restriction point 170 during a biopsy retrieval procedure, as illustrated in FIG. 1. FIG. 1 also illustrates a distal restriction point 180 which exposes the needle 140 beyond the tubular blade 120 after a biopsy retrieval procedure, as described herein with reference to FIG. 2.

After the biopsy punch 100 is inserted into tissue of the subject patient or plant, a withdrawer/ejector 161 withdraws cytopathology tissue into the needle 140 and into the cytopathology specimen chamber 150. The withdrawer/ejector 161 is positioned within the plunger body 160. A distal end of the withdrawer/ejector 161 includes a withdrawer/ejector piston 161a, which is tightly fitted within the interior of the plunger body 160. The withdrawer/ejector piston 161a creates a vacuum within the plunger body 160 when the withdrawer/ejector 161 is withdrawn in a proximal direction. The vacuum withdraws tissue through the needle 140 and into the cytopathology specimen chamber 150. The roughened needle portion 140b holds the retrieved histopathology specimen secure within the tubular blade 120 while the specimen is excised from the subject patient or plant.

FIG. 2 is a cross-sectional view of a biopsy punch 100 in a biopsy release position. Movement of the plunger body 160 is made by grasping the flanged end 162 of the plunger body 160 and moving the flanged end 162 of the plunger body 160 back and forth in a proximal/distal direction. The plunger body 160, the cytopathology specimen chamber 150, the plunger distal end 145, the restriction piston 175, and the needle 140 are configured to uniformly slide together within the punch body 110 of the biopsy punch 100. The restriction piston 175 is moved, via compression of the plunger body 160 from the proximal restriction point 170 to the distal restriction point 180 to expose the needle 140 and the attached histopathology specimen. The roughened needle portion 140b holds the retrieved histopathology specimen secure until it is ready to be removed for testing. In an embodiment, the punch body 110 is transparent in order to view activity of the plunger body 160.

The withdrawer/ejector 161 ejects the cytopathology specimen out of the needle 140, which can be ejected onto a microscopic slide for test preparation and examination. The withdrawer/ejector piston 161a of the withdrawer/ejector 161 forces tissue out of the cytopathology specimen chamber 150 and through the needle 140. The amount of cytopathology specimen ejected through the needle 140 is controlled by further compression of the withdrawer/ejector 161 in a distal direction. Additional cytopathology specimen held within the cytopathology specimen chamber 150 can also be ejected, via compression of the withdrawer/ejector 161 within the plunger body 160.

FIG. 3 is a cross-sectional view of a biopsy punch 100 in a position in which the cytopathology specimen is ejected. The distal end of the punch body 110 is held with one hand, while the other hand moves the withdrawer/ejector 161 in a distal direction. The withdrawer/ejector piston 161a is butted up against the plunger distal end 145 when there is no cytopathology specimen remaining within the cytopathology specimen chamber 150.

In one embodiment, the histopathology specimen is removed first from the tubular blade 120 using movement of the plunger body 160 towards the distal restriction point 180. The histopathology sample can then be removed. The withdrawer/ejector 161 is subsequently used to eject the cytopathology sample from the needle 140 for examination and testing. Ejection of the cytopathology sample prior to removing the histopathology sample can result in some of the cytopathology sample being lost at the edge of the histopathology sample.

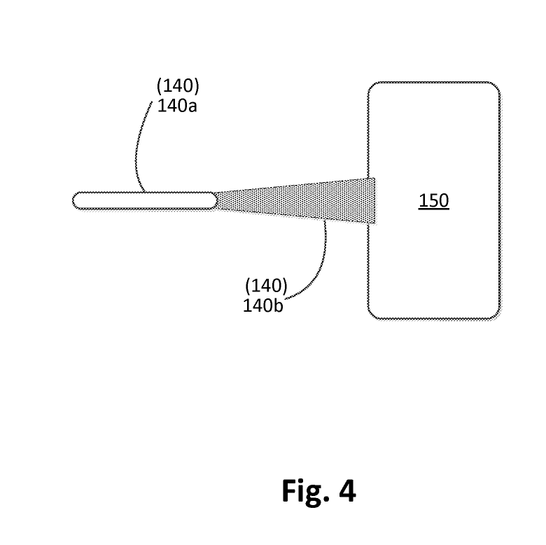

FIG. 4 is a cross-sectional detailed view of needle 140, which includes the smooth needle portion 140a and the roughened needle portion 140b. FIG. 4 illustrates the roughened needle portion 140b tapering from a narrow diameter adjacent to the smooth needle portion 140 and increasing in diameter towards the cytopathology specimen chamber 150. The increase in diameter of the roughened needle portion 140b is exaggerated for illustrative purposes only. The roughened needle portion 140b can be formed by chemically etching the metal surface of the needle 140, mechanically roughening the metal surface, or adhering another material to the metal surface, for example. Other methods of roughening the metal surface are contemplated by embodiments described herein.

The proximal end of the needle 140 is fixed to the plunger distal end 145 and opens into the cytopathology specimen chamber 150. When the biopsy punch 100 is inserted into the tissue to retrieve a tissue sample, tissue is forced into the needle 140, as well as the tubular blade 120. Excess retrieved tissue is forced through the needle 140 and into the cytopathology specimen chamber 150. In addition, cytopathology tissue can be withdrawn into the cytopathology specimen chamber 150, via the withdrawer/ejector 161 within the plunger body 160.

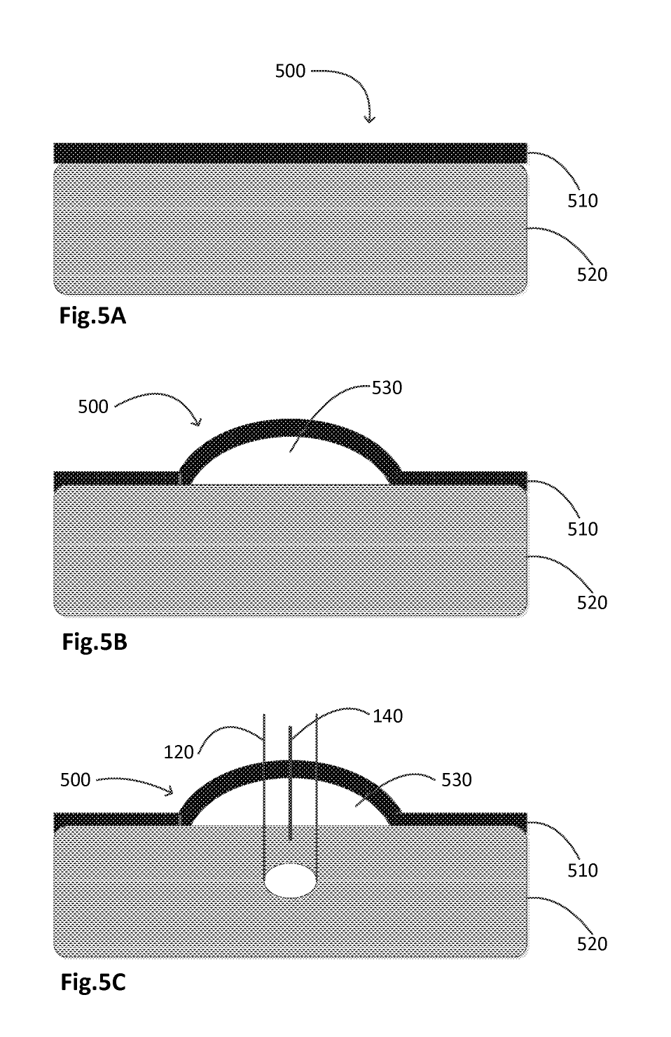

Embodiments described herein for a biopsy punch 100 provide an advantage over conventional biopsy punches by retaining the roof of a specimen of bullous diseases, in which a vesicle separates the tissue into two portions. FIG. 5A is a cross-sectional view of a normal skin tissue 500 in which a thin epidermis external layer 510 overlays a thicker dermis internal layer 520.

FIG. 5B is a cross-sectional view of a vesicle 530 formed between the epidermis external layer 510 and the dermis internal layer 520. It is desirable to maintain the thin superficial roof of the epidermis external layer 510 of the vesicle 530 during a biopsy procedure.

FIG. 5C is a cross-sectional view of the tubular blade 120 of the biopsy punch 100 penetrating through the vesicle 530 into the dermis internal layer 520. The needle 140 of the biopsy punch 100 maintains the thin superficial roof of the epidermis external layer 510 of the vesicle 530 during the biopsy procedure. In a conventional biopsy punch, the thin superficial roof of the epidermis external layer 510 could be damaged or lost during a biopsy procedure.

The superficial thin layer of the biopsy sample is not damaged when using devices and methods described herein. In a conventional biopsy punch, the superficial thin layer can be lost in the surgical field during the biopsy procedure. The biopsy sample can also become lodged within the tubular blade, which can be difficult to extract. The biopsy punch 100 described herein ensures retaining and retrieving an undamaged histopathology specimen and a cytopathology specimen in bullous diseases.

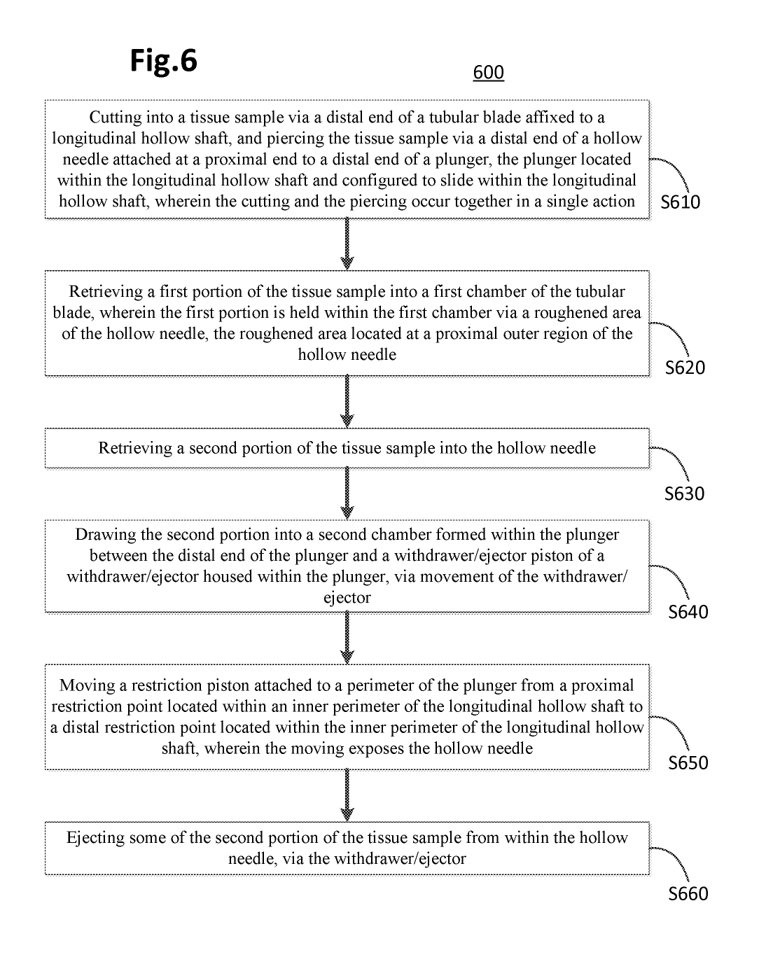

FIG. 6 is a flowchart illustrating a method 600 of using a biopsy punch. Method 600 includes retrieving multiple biopsies in a single action, and disseminating the multiple biopsies for subsequent testing and examination.

In step S610, a tissue sample is cut via a distal end of a tubular blade affixed to a longitudinal hollow shaft, and piercing the tissue sample via a distal end of a hollow needle attached at a proximal end to a distal end of a plunger. The plunger is located within the longitudinal hollow shaft and is configured to slide within the longitudinal hollow shaft. The cutting and the piercing occur together in a single action.

In step S620, a first portion of the tissue sample is retrieved into a first chamber of the tubular blade. The first portion is held within the first chamber via a roughened area of the hollow needle. The roughened area is located at a proximal outer region of the hollow needle.

In step S630, a second portion of the tissue sample is retrieved into the hollow needle. The retrieving can occur, via movement of a withdrawer/ejector piston of a withdrawer/ejector housed within the plunger and/or by forced pressure into the hollow needle during the retrieving.

In step S640, the second portion is drawn into a second chamber formed within the plunger between the distal end of the plunger and the withdrawer/ejector piston, via movement of the withdrawer/ejector. The second chamber can serve as a storage chamber for excess biopsy tissue of the second portion.

In step S650, a restriction piston attached to a perimeter of the plunger is moved from a proximal restriction point located within an inner perimeter of the longitudinal hollow shaft to a distal restriction point located within the inner perimeter of the longitudinal hollow shaft. The moving exposes the hollow needle.

In step S660, some of the second portion of the tissue sample is ejected from within the hollow needle, via the withdrawer/ejector. The amount of the second portion to be ejected can be controlled and can vary. A small amount within the hollow needle can be ejected or the entire contents of the second chamber can be emptied through the hollow needle.

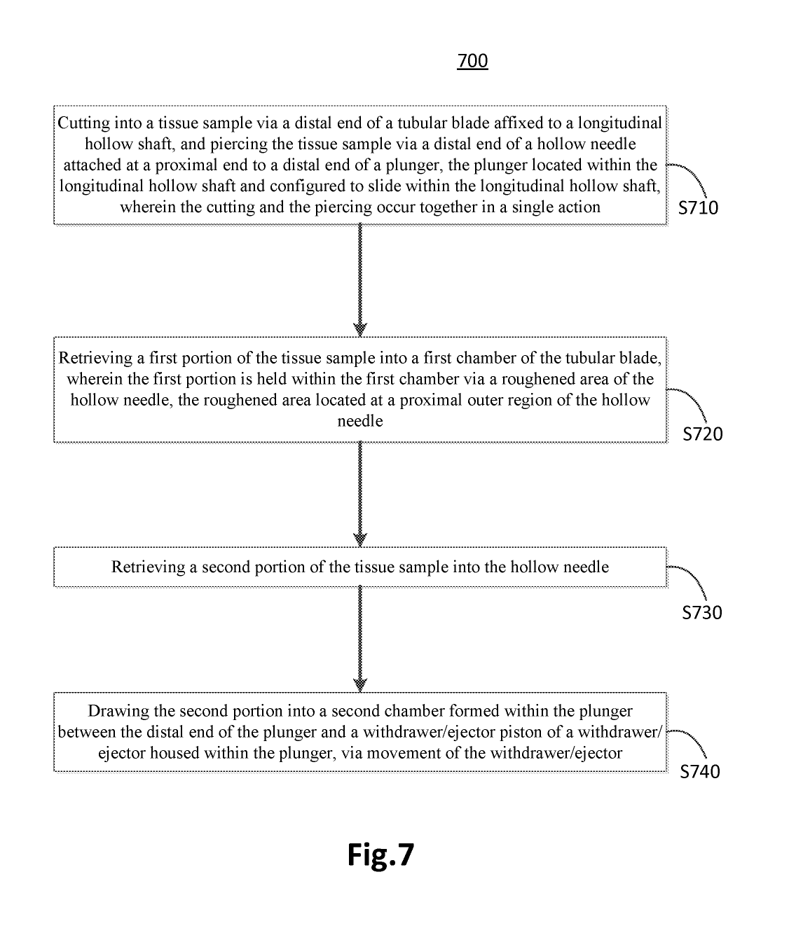

FIG. 7 is a flowchart illustrating a method 700 of obtaining a biopsy specimen. In step S710, a tissue sample is cut via a distal end of a tubular blade affixed to a longitudinal hollow shaft, and piercing the tissue sample via a distal end of a hollow needle attached at a proximal end to a distal end of a plunger. The plunger is located within the longitudinal hollow shaft and is configured to slide within the longitudinal hollow shaft. The cutting and the piercing occur together in a single action.

In step S720, a first portion of the tissue sample is retrieved into a first chamber of the tubular blade. The first portion is held within the first chamber via a roughened area of the hollow needle. The roughened area is located at a proximal outer region of the hollow needle.

In step S730, a second portion of the tissue sample is retrieved into the hollow needle. The retrieving can occur, via movement of a withdrawer/ejector piston connected to a withdrawer/ejector housed within the plunger and/or by forced pressure into the hollow needle during the retrieving.

In step S740, the second portion is drawn into a second chamber formed within the plunger between the distal end of the plunger and the withdrawer/ejector piston, via movement of the withdrawer/ejector housed within the plunger. The second chamber can serve as a storage chamber for excess biopsy tissue of the second portion.

Embodiments herein describe a dual-purpose biopsy punch in which multiple specimens can be obtained in a single retrieval procedure. In addition, different types of specimens can be obtained. A histopathology specimen can be obtained with the tubular blade and a cytopathology specimen can be obtained with the hollow needle.

Exposure of the hollow needle in the biopsy release position aids in easy removal of the histopathology specimen. In addition, a controlled amount of cytopathology specimen can be ejected from the hollow needle, with additional cytopathology specimen available from the cytopathology specimen chamber.

The dual-purpose biopsy punch avoids the necessity of performing multiple biopsy procedures in order to obtain multiple biopsies. The dual-purpose biopsy punch also enables a bedside cytopathology diagnosis in dermatology clinics. Studies have been published about the sensitivity and the specificity of cytopathology in diagnosing dermatological disorders, such as skin tumors. Embodiments described herein facilitate a bedside diagnosis, such as a Mohs Lab in dermatology clinics using the cytopathology sample, which also allows a physician to process a histopathology sample to confirm the cytopathology diagnosis. In an example, a biopsy specimen from a patient with suspected Basal Cell Carcinoma can use the dual biopsy to form a preliminary diagnosis. The cytopathology sample can be checked against results of the histopathology sample before proceeding with surgery.

In addition, the hollow needle can help in obtaining specimens from fragmented tissue or specimens for a tissue culture. Also, the hollow needle has a tissue retaining ability, which allows for smaller sized biopsies, which results in a better cosmetic outcome. In addition, the content of a cyst can be aspirated and examined using embodiments described herein.

The embodiments described herein include the aspects listed below.

(1) A biopsy punch includes a hollow longitudinal shaft including an inner perimeter proximal restriction point and an inner perimeter distal restriction point; a tubular blade affixed to a distal end of the hollow longitudinal shaft, wherein the tubular blade forms a first chamber; a plunger configured to slide within the hollow longitudinal shaft; a withdrawer/ejector housed within the plunger; a withdrawer/ejector piston affixed to a distal end of the withdrawer/ejector; a restriction piston affixed around a perimeter of the plunger, wherein the restriction piston is configured to limit movement of the plunger at the proximal restriction point and the distal restriction point; a second chamber formed within the plunger between a distal end of the plunger and the withdrawer/ejector piston, wherein the withdrawer/ejector is configured to draw and aspirate contents of the second chamber; and a hollow needle configured to move into and out of the first chamber via the plunger, wherein the hollow needle is affixed at a proximal end to the distal end of the plunger, and wherein the proximal end of the needle extends into the second chamber.

(2) The biopsy punch of (1), wherein the tubular blade is configured to cut through a tissue sample and retrieve a first tissue portion within the first chamber of the tubular blade.

(3) The biopsy punch of either one of (1) or (2), wherein the hollow needle is configured to pierce through the tissue sample and retrieve a second tissue portion within the hollow needle.

(4) The biopsy punch of any one of (1) through (3), wherein the needle withdrawer/ejector is configured to draw the second tissue portion of the tissue sample into the hollow needle and to eject the second tissue portion of the tissue sample out of the hollow needle.

(5) The biopsy punch of any one of (1) through (4), wherein the hollow needle has a non-smooth surface area located at a proximal outer region of the hollow needle.

(6) The biopsy punch of any one of (1) through (5), wherein the non-smooth surface area of the hollow needle includes one of a chemically-etched metal surface, a mechanically-roughened metal surface, or a second material adhered to a metal surface of the hollow needle.

(7) The biopsy punch of any one of (1) through (6), wherein the withdrawer/ejector piston is configured to draw contents of the second tissue portion into the second chamber.

(8) The biopsy punch of any one of (1) through (7), wherein the withdrawer/ejector piston is configured to evacuate contents of the second chamber through the hollow needle.

(9) The biopsy punch of any one of (1) through (8), wherein the tubular blade has a sharpened distal edge configured to retrieve a histopathology tissue sample into the first chamber.

(10) The biopsy punch of any one of (1) through (9), wherein the hollow needle has a sharpened distal end configured to retrieve a cytopathology tissue sample.

(11) The biopsy punch of any one of (1) through (10), wherein the hollow needle is centrally located within the tubular blade when the biopsy punch is in a biopsy retrieval position.

(12) The biopsy punch of any one of (1) through (11), wherein the hollow needle is centrally located within the tubular blade when the restriction piston is located at the inner perimeter proximal restriction point.

(13) The biopsy punch of any one of (1) through (12), wherein the hollow needle is located outside of the tubular blade when the biopsy punch is in a biopsy release position.

(14) The biopsy punch of any one of (1) through (13), wherein the hollow needle is located outside of the tubular blade when the restriction piston is located at the inner perimeter distal restriction point.

(15) A method of using a biopsy punch includes cutting into a tissue sample via a distal end of a tubular blade affixed to a longitudinal hollow shaft, and piercing the tissue sample via a distal end of a hollow needle attached at a proximal end to distal end of a plunger, the plunger located within the longitudinal hollow shaft, wherein the cutting and the piercing occur together in a single action; retrieving a first portion of the tissue sample into a first chamber of the tubular blade, wherein the first portion is held within the first chamber via a roughened area of the hollow needle, the roughened area located at a proximal outer region of the hollow needle; retrieving a second portion of the tissue sample into the hollow needle; drawing the second portion into a second chamber located within the plunger at the distal end of the plunger, via a needle withdrawer/ejector housed within the plunger; moving a restriction piston attached to a perimeter of the plunger from a proximal restriction point located within an inner perimeter of the longitudinal hollow shaft to a distal restriction point located within the inner perimeter of the longitudinal hollow shaft, wherein the moving exposes the hollow needle; evacuating some of the second portion of the tissue sample from the second chamber into the hollow needle; and ejecting some of the second portion of the tissue sample from within the hollow needle, via the needle withdrawer/ejector.

(16) The method of (15), wherein retrieving the first portion includes retrieving a histopathology tissue sample into the first chamber of the tubular blade.

(17) The method of either one of (15) or (16), wherein retrieving the second portion includes retrieving a cytopathology tissue sample into the hollow needle, wherein retrieving the first portion and retrieving the second portion occur simultaneously.

(18) The method of any one of (15) through (17), wherein evacuating some of the second portion of the tissue sample from the second chamber includes evacuating via movement of the withdrawer/ejector.

(19) The method of any one of (15) through (18), wherein the hollow needle is centrally located within the first chamber when retrieving the first portion and the second portion of the tissue sample.

(20) The method of any one of (15) through (19), wherein moving the piston from the proximal restriction point to the distal restriction point exposes the retrieved first portion of the tissue sample.

A number of implementations have been described. Nevertheless, it will be understood that various modifications may be made without departing from the spirit and scope of this disclosure. For example, preferable results may be achieved if the steps of the disclosed techniques were performed in a different sequence, if components in the disclosed systems were combined in a different manner, or if the components were replaced or supplemented by other components. Accordingly, other implementations are within the scope that may be claimed.

The foregoing discussion discloses and describes merely exemplary embodiments of the present disclosure. As will be understood by those skilled in the art, the present disclosure may be embodied in other specific forms without departing from the spirit or essential characteristics thereof. Accordingly, the present disclosure is intended to be illustrative and not limiting thereof. The disclosure, including any readily discernible variants of the teachings herein, defines in part, the scope of the foregoing claim terminology.

1. A biopsy punch, comprising:
a hollow longitudinal shaft including an inner perimeter proximal restriction point and an inner perimeter distal restriction point;
a tubular blade affixed to a distal end of the hollow longitudinal shaft, wherein the tubular blade forms a first chamber;
a plunger configured to slide within the hollow longitudinal shaft;
a withdrawer/ejector housed within the plunger;
a withdrawer/ejector piston affixed to a distal end of the withdrawer/ejector;
a restriction piston affixed around a perimeter of the plunger, wherein the restriction piston is configured to limit movement of the plunger at the proximal restriction point and the distal restriction point;
a second chamber formed within the plunger between a distal end of the plunger and the withdrawer/ejector piston, wherein the withdrawer/ejector is configured to draw and aspirate contents of the second chamber; and
a hollow needle configured to move into and out of the first chamber via the plunger, wherein the hollow needle is affixed at a proximal end to the distal end of the plunger, and wherein the proximal end of the needle extends into the second chamber.

2. The biopsy punch of claim 1, wherein the tubular blade is configured to cut through a tissue sample and retrieve a first tissue portion within the first chamber of the tubular blade.

3. The biopsy punch of claim 2, wherein the hollow needle is configured to pierce through the tissue sample and retrieve a second tissue portion within the hollow needle.

4. The biopsy punch of claim 3, wherein the withdrawer/ejector is configured to draw the second tissue portion of the tissue sample into the hollow needle and to eject the second tissue portion of the tissue sample out of the hollow needle.

5. The biopsy punch of claim 4, wherein the hollow needle has a non-smooth surface area located at a proximal outer region of the hollow needle.

6. The biopsy punch of claim 5, wherein the non-smooth surface area of the hollow needle includes one of a chemically-etched metal surface, a mechanically-roughened metal surface, or a second material adhered to a metal surface of the hollow needle.

7. The biopsy punch of claim 1, wherein the withdrawer/ejector piston is configured to draw contents of the second tissue portion into the second chamber.

8. The biopsy punch of claim 1, wherein the withdrawer/ejector piston is configured to evacuate contents of the second chamber through the hollow needle.

9. The biopsy punch of claim 1, wherein the tubular blade has a sharpened distal edge configured to retrieve a histopathology tissue sample into the first chamber.

10. The biopsy punch of claim 1, wherein the hollow needle has a sharpened distal end configured to retrieve a cytopathology tissue sample.

11. The biopsy punch of claim 1, wherein the hollow needle is centrally located within the tubular blade when the biopsy punch is in a biopsy retrieval position.

12. The biopsy punch of claim 11, wherein the hollow needle is centrally located within the tubular blade when the restriction piston is located at the inner perimeter proximal restriction point.

13. The biopsy punch of claim 1, wherein the hollow needle is located outside of the tubular blade when the biopsy punch is in a biopsy release position.

14. The biopsy punch of claim 13, wherein the hollow needle is located outside of the tubular blade when the restriction piston is located at the inner perimeter distal restriction point.

15. A method of using a biopsy punch, the method comprising:
cutting into a tissue sample via a distal end of a tubular blade affixed to a longitudinal hollow shaft, and piercing the tissue sample via a distal end of a hollow needle attached at a proximal end to a distal end of a plunger, the plunger located within the longitudinal hollow shaft and configured to slide within the longitudinal hollow shaft, wherein the cutting and the piercing occur together in a single action;
retrieving a first portion of the tissue sample into a first chamber of the tubular blade, wherein the first portion is held within the first chamber via a roughened area of the hollow needle, the roughened area located at a proximal outer region of the hollow needle;
retrieving a second portion of the tissue sample into the hollow needle;
drawing the second portion into a second chamber formed within the plunger between the distal end of the plunger and a withdrawer/ejector piston of a withdrawer/ejector housed within the plunger, via movement of the withdrawer/ejector;
moving a restriction piston attached to a perimeter of the plunger from a proximal restriction point located within an inner perimeter of the longitudinal hollow shaft to a distal restriction point located within the inner perimeter of the longitudinal hollow shaft, wherein the moving exposes the hollow needle; and
ejecting some of the second portion of the tissue sample from within the hollow needle, via the withdrawer/ejector.

16. The method of claim 15, wherein retrieving the first portion includes retrieving a histopathology tissue sample into the first chamber of the tubular blade.

17. The method of claim 16, wherein retrieving the second portion includes retrieving a cytopathology tissue sample into the hollow needle, wherein retrieving the first portion and retrieving the second portion occur simultaneously.

18. The method of claim 15, wherein evacuating some of the second portion of the tissue sample from the second chamber includes evacuating via movement of the withdrawer/ejector.

19. The method of claim 15, wherein the hollow needle is centrally located within the first chamber when retrieving the first portion and the second portion of the tissue sample.

20. The method of claim 15, wherein moving the restriction piston from the proximal restriction point to the distal restriction point exposes the retrieved first portion of the tissue sample.
